(19) United States
(12) Patent Application Publication
(10) Pub. No.: US 2026/0263542 A1
(43) Pub. Date: Sep. 10, 2026

(54) COMPOSITION CONTAINING PROBIOTICS AND USE THEREOF IN PREPARATION OF WEIGHT-LOSS PRODUCTS

(71) Applicant: GUANGDONG ZHENGDANGNIAN BIOTECHNOLOGY CO.,LTD, GUANGZHOU (CN)

(72) Inventors: TAO WANG, GUANGZHOU (CN); XIANZHI JIANG, GUANGZHOU (CN); WEI WANG, GUANGZHOU (CN); XINJUN LU, GUANGZHOU (CN)

(21) Appl. No.: 19/460,317

(22) Filed: Jan. 27, 2026

(30) Foreign Application Priority Data
Mar. 10, 2025 (CN) 202510272748.5

(51) Int. Cl. A61K 35/747; A61K 35/00; A61K 35/745; A61K 36/185; A61K 36/48; A61K 36/734; A61K 36/752; A61K 36/82; A61K 36/8945; A61K 47/26; A61P 3/04

(52) U.S. Cl. CPC A61K 35/747; A61K 35/745; A61K 36/185; A61K 36/48; A61K 36/734; A61K 36/752; A61K 36/82; A61K 36/8945; A61K 47/26; A61P 3/04; A61K 2035/115; A61K 2236/15; A61K 2236/19; A61K 2236/331; A61K 2236/51; A61K 2236/53

(57) ABSTRACT

The present invention belongs to the technical field of health supplements, and specifically relates to a composition containing probiotics. The composition containing probiotics includes the following components in parts by weight: 5-10 parts of a mixed enzymolysis product, 2-3 parts of Lactobacillus acidophilus, 1-2 parts of Bifidobacterium lactis, 1-2 parts of Lactobacillus bulgaricus, 1-2 parts of Lactobacillus brevis, 1-3 parts of Lactobacillus delbrueckii, 1-2 parts of mannitol, 1-2 parts of isomaltooligosaccharide, and 3-7 parts of a mixed fermentation product. The composition prepared in the present invention exhibits excellent weight-loss effects, particularly demonstrating significant effects for a population having a body weight greater than 80 kg.

### TECHNICAL FIELD

The present invention belongs to the technical field of health supplements, and specifically relates to a composition containing probiotics and use thereof in preparation of weight-loss products.

### BACKGROUND

The demand for weight loss primarily arises from a combination of health, appearance, and psychological considerations. From a health perspective, overweight or obesity constitutes a risk factor for a variety of chronic diseases, including cardiovascular diseases, type 2 diabetes, metabolic syndrome, certain cancers, and musculoskeletal disorders. Obesity further increases the risk of respiratory problems, such as sleep apnea, and imposes additional stress on joints, thereby affecting quality of life.

Weight-loss health supplements provide a convenient option for individuals seeking to support the weight loss process through auxiliary means. Although a healthy diet and regular exercise form the foundation of weight management, certain individuals may be unable to achieve desired weight loss outcomes solely through lifestyle modifications due to time constraints, metabolic factors, or other limitations. In such cases, weight-loss supplements can serve as a supplementary tool to assist in regulating metabolism, suppressing appetite, promoting fat oxidation, or reducing fat absorption. For example, certain health supplements contain natural ingredients, such as green tea extract, L-carnitine, and conjugated linoleic acid, which are considered to facilitate fat metabolism or inhibit fat accumulation. In addition, weight-loss supplements can provide vitamins and minerals to ensure that essential nutrients are not deficient during dietary control. However, it should be noted that weight-loss supplements cannot replace a healthy lifestyle. The function of the weight-loss supplements is generally auxiliary and should be used under professional guidance to ensure safety and efficacy. Generally, the significance of weight-loss supplements lies in providing a scientifically reasonable auxiliary means for individuals seeking additional support during weight loss, thereby facilitating the achievement of weight loss goals and maintaining long-term healthy body weight. Although various weight-loss products are currently available on the market, the efficacy of these products often fails to meet the requirements of consumers.

The primary reason for difficulty in weight loss among a large population with obesity is a reduced metabolic rate. Long-term obesity causes a decrease in basal metabolic rate (BMR), which indicates that the body expends less energy even at rest. As body weight increases, the body gradually adapts to high caloric intake, leading to a slower metabolism. This metabolic adaptation makes weight loss more difficult because even with reduced food intake, the body decreases energy expenditure to maintain existing body weight.

Therefore, there is an urgent need for a composition containing probiotics and use thereof in preparation of weight-loss products

### SUMMARY

An objective of the present invention is to provide a composition containing probiotics and use thereof in preparation of weight-loss products.

To achieve the above objective, the present invention provides the following technical solutions.

A composition containing probiotics includes the following components in parts by weight: 5-10 parts of a mixed enzymolysis product, 2-3 parts of Lactobacillus acidophilus, 1-2 parts of Bifidobacterium lactis, 1-2 parts of Lactobacillus bulgaricus, 1-2 parts of Lactobacillus brevis, 1-3 parts of Lactobacillus delbrueckii, 1-2 parts of mannitol, 1-2 parts of isomaltooligosaccharide, and 3-7 parts of a mixed fermentation product.

Further, a method for preparing the mixed enzymolysis product includes the following steps:
(1) crushing white kidney beans, green tea, and Chinese yam to a particle size of less than 100 mesh to obtain an enzymolysis raw material;
(2) mixing the enzymolysis raw material and water at a weight ratio of 1:(8-10) to obtain an enzymolysis solution, adding cellulase to the enzymolysis solution, and performing enzymolysis for 40-50 min; adjusting pH to 9-10 using an aqueous solution of NaOH at 0.5-1 mol/L, adding alkaline protease, and performing enzymolysis for 40-50 min; subsequently, adjusting pH to 8-9 using an aqueous solution of HCl at 0.5-1 mol/L, adding bromelain, and continuing enzymolysis for 30-40 min; then adjusting pH to 5-6 using an aqueous solution of HCl at 0.5-1 mol/L, adding pectinase, and continuing enzymolysis for 60-80 min; obtaining an enzymolysis product after enzyme deactivation; and
(3) centrifuging the enzymolysis product, collecting a supernatant, filtering the supernatant through an ultrafiltration membrane to obtain ultrafiltrate, concentrating and spray-drying the ultrafiltrate to obtain the mixed enzymolysis product.

Further, the white kidney beans, the green tea, and the Chinese yam are mixed at a weight ratio of (0.3-0.5):1:(1.4-1.7).

Further, the cellulase is added to the enzymolysis solution at an amount of 2000-3000 U/mL, the alkaline protease is added to the enzymolysis solution at an amount of 1000-2000 U/mL, the bromelain is added to the enzymolysis solution at an amount of 1000-2000 U/mL, and the pectinase is added to the enzymolysis solution at an amount of 1000-2000 U/mL.

Further, the supernatant is filtered through an ultrafiltration membrane having a molecular weight of 4000-5000 Da.

Further, a method for preparing the mixed fermentation product includes the following steps:
(1) crushing lotus leaves, hawthorn, and citrus to a particle size of less than 100 mesh to obtain a mixed raw material;
(2) subjecting the mixed raw material to low temperature at −30° C. for 2-3 min, rapidly increasing the temperature to 23-25° C. within 2 s, and repeating the temperature cycling 2-3 times; subsequently pressurizing to 20-25 MPa, maintaining for 3-5 min, depressurizing to atmospheric pressure, and repeating the pressurization cycle 2-3 times to obtain a pretreated mixed raw material;
(3) mixing the pretreated mixed raw material and water at a weight ratio of 1:(8-10) to obtain a mixed solution, adding a composite bacterial culture to the mixed solution, performing anaerobic fermentation for 10-15 h, and sterilizing to obtain a fermentation broth; and
(4) centrifuging the fermentation broth, collecting a supernatant, filtering the supernatant through an ultrafiltration membrane having a molecular weight of 3000-5000 Da to obtain ultrafiltrate, concentrating and spray-drying the ultrafiltrate to obtain the mixed fermentation product.

Further, the lotus leaves, the hawthorn, and the citrus are mixed at a weight ratio of 1:(1.2-1.5):(0.4-0.7).

Further, the composite bacterial culture includes Lactobacillus casei, Lactobacillus brevis, and Bifidobacterium longum; where the Lactobacillus casei is added at 10⁷-10⁸ CFU/mL of the mixed solution, the Lactobacillus brevis is added at 10⁹-10¹⁰ CFU/mL of the mixed solution, and the Bifidobacterium longum is added at 10⁸-10⁹ CFU/mL of the mixed solution.

Further, a method for preparing the composition containing probiotics includes the following steps: weighing the components according to a weight ratio and mixing uniformly to obtain the composition containing probiotics.

The present invention provides use of the composition containing probiotics in preparation of weight-loss products.

Compared with the prior art, the present invention has the following advantages and beneficial effects:

1. In the mixed enzymolysis product prepared by enzymolysis, white kidney beans contain abundant α-amylase inhibitors, which inhibit the activity of α-amylase in the small intestine and delay the digestion and absorption of carbohydrates. This facilitates the prevention of rapid blood glucose increase, reduces insulin secretion, and avoids further aggravation of insulin resistance. By inhibiting α-amylase, white kidney bean extract can reduce postprandial blood glucose spikes, stabilize blood glucose levels, and decrease the occurrence of hunger and overeating. Catechins (such as EGCG) in green tea exhibit strong antioxidant activity, activate lipolytic enzymes in adipocytes, and promote fat oxidation and metabolism. Studies indicate that green tea extract can increase energy expenditure and reduce fat accumulation, particularly in the abdominal region. Polyphenols in green tea further improve insulin sensitivity, facilitate more efficient glucose utilization, and reduce fat storage. In addition, green tea exhibits anti-inflammatory effects, which can alleviate chronic inflammation associated with obesity and further improve metabolic health. Polysaccharides in Chinese yam improve insulin sensitivity, facilitate more efficient glucose utilization, and reduce fat storage. In addition, Chinese yam exhibits anti-inflammatory effects, which can alleviate chronic inflammation associated with obesity and further improve symptoms of metabolic syndrome.

2. In the composition of the present invention, lotus leaf, hawthorn, and citrus are mixed, and the mixture is fermented using specific bacteria. The resulting product assists a population having a body weight greater than 80 kg in reducing body weight through multiple mechanisms. The lotus leaves contain abundant alkaloids (such as nuciferine), which inhibit the absorption of fats in the intestine and reduce fat accumulation in the body. During fermentation, specific bacteria further decompose complex components in lotus leaves, releasing additional active substances and enhancing the inhibition of fat absorption. Flavonoids in lotus leaves exhibit antioxidant activity, activate lipolytic enzymes in adipocytes, and promote fat oxidation and metabolism. The fermentation process increases the bioavailability of these flavonoids, facilitating absorption by the body and accelerating fat burning.

The hawthorn contains abundant organic acids (such as chlorogenic acid and caffeic acid) and lipolytic enzymes, which promote gastric juice secretion and accelerate digestion, particularly of fatty foods. During fermentation, specific bacteria further decompose organic acids in hawthorn, producing additional metabolites and enhancing the promotion of digestion and fat metabolism. The flavonoids and triterpenoids in hawthorn reduce blood lipids by lowering triglyceride and low-density lipoprotein (LDL) cholesterol levels, thereby reducing the risk of atherosclerosis. The fermentation process increases the bioactivity of these compounds and enhances lipid-lowering effects. The citrus contains abundant vitamin C and flavonoids (such as hesperidin and naringin), which exhibit strong antioxidant activity, activate fat-oxidizing enzymes in adipocytes, and promote fat oxidation and metabolism. During fermentation, specific bacteria further decompose flavonoids in citrus, releasing additional active substances and enhancing the promotion of fat oxidation.

Specific bacteria produce short-chain fatty acids (SCFAs), such as acetate, propionate, and butyrate, during fermentation. These SCFAs, serving as an energy source, regulate the balance of intestinal microbiota, promote the growth of beneficial bacteria, and inhibit the proliferation of harmful bacteria. A healthy intestinal microbiota improves metabolic health, reduces inflammatory responses, and promotes fat breakdown and metabolism. SCFAs generated during fermentation further enhance intestinal barrier function, reduce the absorption of harmful substances, and prevent intestinal permeability. This contributes to improved intestinal health, reduction of chronic inflammation associated with obesity, and further promotion of fat burning.

3. The present invention combines Lactobacillus acidophilus, Bifidobacterium lactis, Lactobacillus bulgaricus, Lactobacillus brevis, and Lactobacillus delbrueckii, thereby significantly improving weight-loss effects. The combined bacterial consortium promotes fat metabolism, improves intestinal health, regulates blood glucose and insulin sensitivity, enhances satiety, and reduces appetite through multiple synergistic mechanisms. Lactobacillus acidophilus produces short-chain fatty acids (SCFAs), particularly acetate and propionate, which inhibit fatty acid synthase in adipocytes and reduce fat synthesis and storage. In addition, Lactobacillus acidophilus regulates intestinal microbiota, reduces endotoxins produced by harmful bacteria, lowers chronic inflammation, and promotes fat breakdown. Bifidobacterium lactis activates lipolytic enzymes in adipocytes, promotes fat breakdown and metabolism, and facilitates more efficient fat consumption by the body. Lactobacillus bulgaricus inhibits the growth of harmful bacteria through competitive exclusion, maintains intestinal microbiota balance, reduces endotoxins produced by harmful bacteria, lowers chronic inflammation, and promotes fat breakdown. Lactobacillus brevis enhances intestinal barrier function, reduces the absorption of harmful substances, and prevents intestinal permeability by producing mucins and tight junction proteins. This contributes to improved intestinal health, reduction of chronic inflammation associated with obesity, and further promotion of fat burning.

### DETAILED DESCRIPTION OF THE EMBODIMENTS

The following clearly and completely describes the technical solutions in the embodiments of the present invention. It is clear that the described embodiments are merely a part rather than all of the embodiments of the present invention. All other embodiments obtained by those of ordinary skill in the art based on embodiments of the present invention without creative efforts shall fall within the protection scope of the present invention.

### Example 1

This example provides a composition containing probiotics, including the following components in parts by weight: 5 parts of a mixed enzymolysis product, 2 parts of Lactobacillus acidophilus, 1 part of Bifidobacterium lactis, 1 part of Lactobacillus bulgaricus, 1 part of Lactobacillus brevis, 1 part of Lactobacillus delbrueckii, 1 part of mannitol, 1 part of isomaltooligosaccharide, and 3 parts of a mixed fermentation product.

The mixed enzymolysis product was prepared by the following steps:
(1) White kidney beans, green tea, and Chinese yam were crushed to a particle size of less than 100 mesh to obtain an enzymolysis raw material.
(2) The enzymolysis raw material and water were mixed at a weight ratio of 1:8 to obtain an enzymolysis solution, cellulase was added to the enzymolysis solution, and enzymolysis was performed for 40 min; the pH value was adjusted to 9 using an aqueous solution of NaOH at a concentration of 0.5 mol/L, alkaline protease was added, and enzymolysis was performed for 40 min; the pH value was then adjusted to 8 using an aqueous solution of HCl at a concentration of 0.5 mol/L, bromelain was added, and enzymolysis was continued for 30 min; the pH value was further adjusted to 5 using an aqueous solution of HCl at a concentration of 0.5 mol/L, pectinase was added, and enzymolysis was continued for 60 min; after enzyme inactivation, an enzymolysis product was obtained.
(3) The enzymolysis product was centrifuged, and a supernatant was collected. The supernatant was filtered through an ultrafiltration membrane to obtain an ultrafiltrate, which was concentrated and spray-dried to obtain the mixed enzymolysis product.

The white kidney bean, the green tea, and the Chinese yam were mixed at a weight ratio of 0.3:1:1.4.

The cellulase was added to the enzymolysis solution at an amount of 2000 U/mL, the alkaline protease was added to the enzymolysis solution at an amount of 1000 U/mL, the bromelain was added to the enzymolysis solution at an amount of 1000 U/mL, and the pectinase was added to the enzymolysis solution at an amount of 1000 U/mL.

The supernatant was filtered through an ultrafiltration membrane having a molecular weight of 4000 Da.

The mixed fermentation product was prepared by the following steps:
(1) Lotus leaves, hawthorn, and citrus were crushed to a particle size of less than 100 mesh to obtain a mixed raw material.
(2) The mixed raw material was first placed at −30° C. for 2 min, followed by rapid heating to 23° C. within 2 s, and the temperature cycling was repeated twice; the material was then pressurized to 20 MPa and maintained for 3 min, followed by depressurization to atmospheric pressure, and the pressurization cycle was repeated twice; and a pretreated mixed raw material was obtained.
(3) The pretreated mixed raw material and water were mixed at a weight ratio of 1:8 to obtain a mixed solution, to which a composite bacterial culture was added; anaerobic fermentation was performed for 10 h, followed by sterilization to obtain a fermentation broth.
(4) The fermentation broth was centrifuged, and a supernatant was collected. The supernatant was filtered through an ultrafiltration membrane having a molecular weight cutoff of 3000 Da to obtain an ultrafiltrate, and the ultrafiltrate was concentrated and spray-dried to obtain the mixed fermentation product.

The lotus leaves, the hawthorn, and the citrus were mixed at a weight ratio of 1:1.2:0.4.

The composite bacterial culture includes Lactobacillus casei, Lactobacillus brevis, and Bifidobacterium longum; where the Lactobacillus casei is added at 10⁷ CFU/mL of the mixed solution, the Lactobacillus brevis is added at 10⁹ CFU/mL of the mixed solution, and the Bifidobacterium longum is added at 10⁸ CFU/mL of the mixed solution.

A method for preparing the composition containing probiotics includes the following steps: the components were weighed according to the weight ratio and mixed uniformly to obtain the composition.

### Example 2

This example provides a composition containing probiotics, including the following components in parts by weight: 10 parts of a mixed enzymolysis product, 3 parts of Lactobacillus acidophilus, 2 parts of Bifidobacterium lactis, 2 parts of Lactobacillus bulgaricus, 2 parts of Lactobacillus brevis, 3 parts of Lactobacillus delbrueckii, 2 parts of mannitol, 2 parts of isomaltooligosaccharide, and 7 parts of a mixed fermentation product.

The mixed enzymolysis product was prepared by the following steps:
(1) White kidney beans, green tea, and Chinese yam were crushed to a particle size of less than 100 mesh to obtain an enzymolysis raw material.
(2) The enzymolysis raw material and water were mixed at a weight ratio of 1:10 to obtain an enzymolysis solution, cellulase was added to the enzymolysis solution, and enzymolysis was performed for 50 min; the pH value was adjusted to 10 using an aqueous solution of NaOH at a concentration of 1 mol/L, alkaline protease was added, and enzymolysis was performed for 50 min; the pH value was then adjusted to 9 using an aqueous solution of HCl at a concentration of 1 mol/L, bromelain was added, and enzymolysis was continued for 40 min; the pH value was further adjusted to 6 using an aqueous solution of HCl at a concentration of 1 mol/L, pectinase was added, and enzymolysis was continued for 80 min; after enzyme inactivation, an enzymolysis product was obtained.
(3) The enzymolysis product was centrifuged, and a supernatant was collected. The supernatant was filtered through an ultrafiltration membrane to obtain an ultrafiltrate, which was concentrated and spray-dried to obtain the mixed enzymolysis product.

The white kidney bean, the green tea, and the Chinese yam were mixed at a weight ratio of 0.5:1:1.7.

The cellulase was added to the enzymolysis solution at an amount of 3000 U/mL, the alkaline protease was added to the enzymolysis solution at an amount of 2000 U/mL, the bromelain was added to the enzymolysis solution at an amount of 2000 U/mL, and the pectinase was added to the enzymolysis solution at an amount of 2000 U/mL.

The supernatant was filtered through an ultrafiltration membrane having a molecular weight of 5000 Da.

The mixed fermentation product was prepared by the following steps:
(1) Lotus leaves, hawthorn, and citrus were crushed to a particle size of less than 100 mesh to obtain a mixed raw material.
(2) The mixed raw material was first placed at −30° C. for 3 min, followed by rapid heating to 25° C. within 2 s, and the temperature cycling was repeated three times; the material was then pressurized to 25 MPa and maintained for 5 min, followed by depressurization to atmospheric pressure, and the pressurization cycle was repeated three times; and a pretreated mixed raw material was obtained.
(3) The pretreated mixed raw material and water were mixed at a weight ratio of 1:10 to obtain a mixed solution, to which a composite bacterial culture was added; anaerobic fermentation was performed for 15 h, followed by sterilization to obtain a fermentation broth.
(4) The fermentation broth was centrifuged, and a supernatant was collected. The supernatant was filtered through an ultrafiltration membrane having a molecular weight cutoff of 5000 Da to obtain an ultrafiltrate, and the ultrafiltrate was concentrated and spray-dried to obtain the mixed fermentation product.

The lotus leaves, the hawthorn, and the citrus were mixed at a weight ratio of 1:1.5:0.7.

The composite bacterial culture includes Lactobacillus casei, Lactobacillus brevis, and Bifidobacterium longum; where the Lactobacillus casei is added at 10⁸ CFU/mL of the mixed solution, the Lactobacillus brevis is added at 10¹⁰ CFU/mL of the mixed solution, and the Bifidobacterium longum is added at 10⁹ CFU/mL of the mixed solution.

A method for preparing the composition containing probiotics includes the following steps: the components were weighed according to the weight ratio and mixed uniformly to obtain the composition.

### Comparative Example 1

The difference between Comparative Example 1 and Example 1 was that a composition containing probiotics includes the following components in parts by weight: 3 parts of a mixed enzymolysis product, 4 parts of Lactobacillus acidophilus, 3 parts of Bifidobacterium lactis, 3 parts of Lactobacillus delbrueckii, 1 part of mannitol, 1 part of isomaltooligosaccharide, and 1 part of a mixed fermentation product.

### Comparative Example 2

The difference between Comparative Example 2 and Example 1 was that the white kidney bean, the green tea, and the Chinese yam were mixed at a weight ratio of 1:1:1.

### Comparative Example 3

The difference between Comparative Example 3 and Example 1 was that the cellulase was added to the enzymolysis solution at an amount of 1000 U/mL, the alkaline protease was added at an amount of 3000 U/mL, the bromelain was added at an amount of 500 U/mL, and the pectinase was added at an amount of 500 U/mL.

### Comparative Example 4

The difference between Comparative Example 4 and Example 1 was that the lotus leaves, the hawthorn, and the citrus were mixed at a weight ratio of 1:1:1.

### Comparative Example 5

The difference between Comparative Example 5 and Example 1 was that the composite bacterial culture includes Lactobacillus casei, Lactobacillus brevis, and Bifidobacterium longum; where the Lactobacillus casei is added at 10⁹ CFU/mL of the mixed solution, the Lactobacillus brevis is added at 10⁸ CFU/mL of the mixed solution, and the Bifidobacterium longum is added at 10⁷ CFU/mL of the mixed solution.

### Performance Testing

### 1. Subjects and Methods

### 1.1 Subjects

All subjects were evaluated for body mass index prior to the experiment, and all were classified as obese. Among 80 subjects, 40 were male and 40 were female, aged 18-50 years, with a mean age of 34.7±7.8 years.

### 1.2 Methods

Subjects were randomly divided into eight groups (N=10 each): negative control group, Example 1, Example 2, Comparative Example 1, Comparative Example 2, Comparative Example 3, Comparative Example 4, and Comparative Example 5. The negative control group was administered a placebo at 2 capsules per day. Subjects in the Example and Comparative Example groups were administered the respective compositions as described above continuously for 8 weeks. During the experiment, diet was not controlled, exercise was not deliberately increased, and other weight-loss foods or drugs were prohibited.

### 1.3 Test Parameters

1.3.1 Body composition testing: Before the experiment and weekly thereafter, body weight, body fat percentage, and body mass index of subjects were measured using a DX200 super-advanced body composition analyzer.

### 1.4 Data Analysis

The obtained data were processed using Excel. Statistical analysis was performed using a single-blind method and t-test. Data were expressed as mean±SD.

The results are shown in Table 1.

TABLE 1
Changes in body weight of subjects before
and after the experiment (kg)

| | Before experiment | After experiment |
|---|---|---|
| | (kg) | (kg) |
| Example 1 | 81.43 ± 12.67 | 74.43 ± 13.81 |
| Example 2 | 80.51 ± 13.22 | 74.51 ± 12.34 |
| Comparative Example 1 | 80.89 ± 12.86 | 77.89 ± 13.57 |
| Comparative Example 2 | 82.24 ± 14.13 | 78.24 ± 13.44 |
| Comparative Example 3 | 81.46 ± 12.35 | 77.46 ± 14.79 |
| Comparative Example 4 | 82.38 ± 13.47 | 75.38 ± 12.83 |
| Comparative Example 5 | 80.03 ± 14.79 | 77.03 ± 13.40 |
| Control group | 80.72 ± 14.01 | 79.52 ± 12.22 |

It can be seen from Table 1 that the composition containing probiotics of the present invention exhibits excellent weight-loss effects.

It can be seen from the comparison between Example 1 and Comparative Example 1 that the weight ratios and composition of the composition of the present invention significantly improve weight-loss effects.

It can be seen from the comparison between Example 1 and Comparative Examples 2-3 that the mixed enzymolysis product prepared by the specific enzymolysis method significantly improves weight-loss effects.

It can be seen from the comparison between Example 1 and Comparative Examples 4-5 that the mixed fermentation product prepared by the specific fermentation method significantly improves weight-loss effects.

The foregoing descriptions are preferred embodiments of the present invention. It should be noted that those of ordinary skill in the art may make some improvements and polishing without departing from the principle of the present invention, and the improvements and polishing shall fall within the protection scope of the present invention.

1. A composition containing probiotics, comprising the following components in parts by weight: 5-10 parts of a mixed enzymolysis product, 2-3 parts of Lactobacillus acidophilus, 1-2 parts of Bifidobacterium lactis, 1-2 parts of Lactobacillus bulgaricus, 1-2 parts of Lactobacillus brevis, 1-3 parts of Lactobacillus delbrueckii, 1-2 parts of mannitol, 1-2 parts of isomaltooligosaccharide, and 3-7 parts of a mixed fermentation product; wherein
a method for preparing the mixed enzymolysis product comprises the following steps:
(1) crushing white kidney beans, green tea, and Chinese yam to a particle size of less than 100 mesh to obtain an enzymolysis raw material;
(2) mixing the enzymolysis raw material and water at a weight ratio of 1:(8-10) to obtain an enzymolysis solution, adding cellulase to the enzymolysis solution, and performing enzymolysis for 40-50 min; adjusting pH to 9-10 using an aqueous solution of NaOH at 0.5-1 mol/L, adding alkaline protease, and performing enzymolysis for 40-50 min; subsequently, adjusting pH to 8-9 using an aqueous solution of HCl at 0.5-1 mol/L, adding bromelain, and continuing enzymolysis for 30-40 min; then adjusting pH to 5-6 using an aqueous solution of HCl at 0.5-1 mol/L, adding pectinase, and continuing enzymolysis for 60-80 min; obtaining an enzymolysis product after enzyme deactivation;
(3) centrifuging the enzymolysis product, collecting a supernatant, filtering the supernatant through an ultrafiltration membrane to obtain ultrafiltrate, concentrating and spray-drying the ultrafiltrate to obtain the mixed enzymolysis product; wherein
the white kidney beans, the green tea, and the Chinese yam are mixed at a weight ratio of (0.3-0.5):1:(1.4-1.7);
the cellulase is added to the enzymolysis solution at an amount of 2000-3000 U/mL, the alkaline protease is added to the enzymolysis solution at an amount of 1000-2000 U/mL, the bromelain is added to the enzymolysis solution at an amount of 1000-2000 U/mL, and the pectinase is added to the enzymolysis solution at an amount of 1000-2000 U/mL;
a method for preparing the mixed fermentation product comprises the following steps:
(1) crushing lotus leaves, hawthorn, and citrus to a particle size of less than 100 mesh to obtain a mixed raw material;
(2) subjecting the mixed raw material to low temperature at −30° C. for 2-3 min, rapidly increasing the temperature to 23-25° C. within 2 s, and repeating the temperature cycling 2-3 times; subsequently pressurizing to 20-25 MPa, maintaining for 3-5 min, depressurizing to atmospheric pressure, and repeating the pressurization cycle 2-3 times to obtain a pretreated mixed raw material;
(3) mixing the pretreated mixed raw material and water at a weight ratio of 1:(8-10) to obtain a mixed solution, adding a composite bacterial culture to the mixed solution, performing anaerobic fermentation for 10-15 h, and sterilizing to obtain a fermentation broth;
(4) centrifuging the fermentation broth, collecting a supernatant, filtering the supernatant through an ultrafiltration membrane having a molecular weight of 3000-5000 Da to obtain ultrafiltrate, concentrating and spray-drying the ultrafiltrate to obtain the mixed fermentation product; wherein
the lotus leaves, the hawthorn, and the citrus are mixed at a weight ratio of 1:(1.2-1.5):(0.4-0.7); and
the composite bacterial culture comprises Lactobacillus casei, Lactobacillus brevis, and Bifidobacterium longum; wherein the Lactobacillus casei is added at 10⁷-10⁸ CFU/mL of the mixed solution, the Lactobacillus brevis is added at 10⁹-10¹⁰ CFU/mL of the mixed solution, and the Bifidobacterium longum is added at 10⁸-10⁹ CFU/mL of the mixed solution.

2. The composition containing probiotics according to claim 1, wherein the supernatant is filtered through an ultrafiltration membrane having a molecular weight of 4000-5000 Da.

3. The composition containing probiotics according to claim 1, wherein a method for preparing the composition containing probiotics comprises the following steps: weighing the components according to a weight ratio and mixing uniformly to obtain the composition containing probiotics.

4. Use of the composition containing probiotics according to claim 1 in preparation of weight-loss products.
